(19) United States
(12) Patent Application Publication
(10) Pub. No.: US 2020/0118644 A1
(43) Pub. Date: Apr. 16, 2020

(54) MICROSATELLITE INSTABILITY DETERMINATION SYSTEM AND RELATED METHODS

(71) Applicant: TEMPUS LABS, INC., Chicago, IL (US)

(72) Inventors: Aly Azeem Khan, Chicago, IL (US); Denise Lau, Chicago, IL (US)

(21) Appl. No.: 16/653,868

(22) Filed: Oct. 15, 2019

Related U.S. Application Data
(60) Provisional application No. 62/745,946, filed on Oct. 15, 2018.

(51) Int. Cl. G16B 20/10; G16B 40/10; G16B 50/30; G16B 50/20; G16B 50/10; G16B 30/20; G16H 50/30; G06F 17/18

(52) U.S. Cl. CPC G16B 20/10; G16B 40/10; G16B 50/30; G06F 17/18; G16B 50/10; G16B 30/20; G16H 50/30; G16B 50/20

(57) ABSTRACT

Methods and systems for determining microsatellite instability (MSI) directly from microsatellite region mappings for specific loci in the genome are provided. Techniques include an MSI assay that may be deployed in a paired form, that is, as tumor sample and matched normal sample MSI assay, or an unpaired form, that is, as a tumor-only MSI assay. The techniques provide an automated process for MSI determination by mapping read counts in tumor samples and normal samples and comparing the two, for an identified set of 43 microsatellite loci.

### CROSS-REFERENCE TO RELATED APPLICATIONS

This application claims benefit of priority to and claims under 35 U.S.C. § 119(e)(1) the benefit of the filing date of U.S. provisional application Ser. No. 62/745,946 filed Oct. 15, 2018, the entire disclosure of which is incorporated herein by reference.

### FIELD OF THE INVENTION

The present disclosure relates to the use of next generation sequencing to determine microsatellite instability (MSI) status.

### BACKGROUND

The background description provided herein is for the purpose of generally presenting the context of the disclosure. Work of the presently named inventors, to the extent it is described in this background section, as well as aspects of the description that may not otherwise qualify as prior art at the time of filing, are neither expressly nor impliedly admitted as prior art against the present disclosure.

Microsatellite instability (MSI) is a clinically actionable genomic indication for cancer immunotherapies. MSI is a type of genomic instability that occurs in repetitive DNA regions and results from defects in DNA mismatch repair. MSI occurs in a variety of cancers. This mismatch repair defect results in a hyper-mutated phenotype where alterations accumulate in the repetitive microsatellite regions of DNA. In Microsatellite Instability-High (MSI-H) tumors, the number of short tandem repeats present in microsatellite regions differ significantly from the number of repeats that are in the DNA of a benign cell.

In clinical MSI PCR testing, tumors with length differences in 2 or more of the 5 microsatellite markers on the Bethesda panel are unstable and considered Microsatellite Instability-High (MSI-H). Microsatellite Stable (MSS) tumors are tumors that have no functional defects in DNA mismatch repair and have no significant differences between tumor and normal in any of the 5 microsatellite regions. Microsatellite Instability-Low (MSI-L) is a tumor with an intermediate phenotype that has 1 unstable marker. Overall, MSI-H is observed in 15% of sporadic colorectal tumors worldwide and has been reported in other cancer types including uterine and gastric cancers.

### SUMMARY OF THE INVENTION

The present application presents techniques for determining microsatellite instability (MSI) directly from microsatellite region mappings for specific loci in the genome. The techniques include an MSI assay that may employ a support vector machine (SVM) classifier to assess MSI. The assay may be a tumor-normal MSI assay in some examples. In other examples, the assay may be a tumor-only MSI assay. The techniques provide an automated process for MSI testing and MSI status prediction via a supervised machine learning process.

In accordance with an example, a computer-implemented method of indicating a likelihood of microsatellite instability comprises: for each locus in a plurality of microsatellite instability (MSI) loci: mapping a first plurality of genomic sequencing reads from a tumor specimen to the locus; mapping a second plurality of genomic sequencing reads from a matched-normal specimen to the locus; comparing the mapping of the first plurality to the mapping of the second plurality and determining the likelihood of microsatellite instability based on the comparison; and generating a report indicating the determined likelihood of microsatellite instability.

In accordance with an example, the plurality of MSI loci includes at least one locus listed in Table 1 below.

In accordance with an example, the plurality of MSI loci includes all of the loci listed in Table 1 below.

In accordance with an example, the plurality of MSI loci includes at least one locus on a chromosome listed in Table 1 below.

In accordance with an example, each locus in the plurality of MSI loci is positioned on a chromosome listed in Table 1 below.

In accordance with an example, mapping the first plurality comprises mapping reads containing 3-6 base pairs, and mapping the second plurality comprises mapping reads containing 3-6 base pairs

In accordance with an example, mapping the first plurality of genomic sequencing reads comprises mapping at least 30-40 genomic sequencing reads from the tumor sample; and mapping the second plurality of genomic sequencing reads comprises mapping at least 30-40 genomic sequencing reads from the normal sample.

In accordance with an example, the computer-implemented method includes when mapping the first plurality of genomic sequencing reads, determining if at least 20-30 microsatellites meet a coverage minimum; and when mapping the second plurality of genomic sequencing reads, determining if at least 20-30 microsatellites meet a coverage minimum.

In accordance with an example, the computer-implemented method includes if at least 20-30 microsatellites do not meet the coverage minimum when mapping the second plurality of genomic sequencing reads, then replacing the mapping of the second plurality of genomic sequencing reads with mean and variance data from a trained sequencing data before performing the comparison.

In accordance with an example, the computer-implemented method includes comparing the mapping of the first plurality to the mapping of the second plurality and determining the likelihood of microsatellite instability based on the comparison by measuring changes in the number of repeat units in the first plurality of genomic sequencing reads from the tumor specimen to the number of repeat units in the second plurality of genomic sequencing reads from the matched-normal specimen

In accordance with an example, the computer-implemented method includes comparing the mapping of the first plurality to the mapping of the second plurality and determining the likelihood of microsatellite instability based on the comparison using a Kolmogorov-Smirov test.

In accordance with an example, the computer-implemented method includes determining the likelihood of microsatellite instability based on a p value (probability value).

In accordance with an example, the computer-implemented method includes determining the likelihood of microsatellite instability as microsatellite instability high (MSI-H), microsatellite stable (MSI-S), or microsatellite equivocal (MSI-E).

In accordance with an example, MSI-H is > about 70% probability, MSI-E is between about 50% and about 70% probability, and MSI-S is < about 50%, where “about” is defined as between 0% to 10% +/− difference.

In accordance with an example, the computer-implemented method includes determining a therapeutic for a subject based on the determined likelihood of microsatellite instability.

In accordance with an example, the therapeutic is selected from the group consisting of fluoropyrimidine, oxaliplatin, irinotecan, Ipilimumab, nivolumab, Pembrolizumab, an anti-PD-L1 antibody (e.g., durvalumab), an anti-CTLA antibody (e.g., tremelimumab), and checkpoint inhibitor (e.g., PD-1 inhibitor, PD-L1 inhibitor, PD-L2 inhibitor, CTLA-4 inhibitor).

In accordance with an example, a computing device is provided to perform the computer-implemented methods herein.

In accordance with an example, a computing device configured to indicate a likelihood of microsatellite instability, the computing device comprising one or more processors configured to: for each locus in a plurality of microsatellite instability (MSI) loci: map a first plurality of genomic sequencing reads from a tumor specimen to the locus; map a second plurality of genomic sequencing reads from a matched-normal specimen to the locus; compare the mapping of the first plurality to the mapping of the second plurality and determine the likelihood of microsatellite instability based on the comparison; and generate a report indicating the determined likelihood of microsatellite instability.

### BRIEF DESCRIPTION OF THE DRAWINGS

The figures described below depict various aspects of the system and methods disclosed herein. It should be understood that each figure depicts an example of aspects of the present systems and methods.

FIG. 1 is a block diagram of an example method of MSI Detection and classification in a paired mode using tumor and normal matched samples, in accordance with an example implementation.

FIG. 2 is a block diagram of an example method of MSI Detection and classification in an unpaired mode using tumor-only samples, in accordance with an example implementation.

FIG. 3 is a plot of validation results for microsatellite status classification from a genomic sequencing assay on a set of tumor samples, in accordance with an example implementation. The plot displays the count of samples (y-axis) and exemplary thresholds of MSI-H, MSE, and MSS (x-axis).

FIG. 4 is a screenshot of an example clinical reporting of MSI status, in accordance with an example implementation.

FIG. 5 illustrates an example computing device for implementing the processes of FIGS. 1 and 2, in accordance with an example implementation.

### DETAILED DESCRIPTION

The present application presents techniques for determining microsatellite instability (MSI) directly from microsatellite region mappings for specific loci in the genome. In some examples, a MSI assay is disclosed. The assay may be a tumor-normal MSI assay. The MSI assay may refer to specific loci in the genome. The MSI assay may employ a support vector machine (SVM) classifier. For a tumor-normal MSI assay, instability may be tested at each locus by comparing the distributions of the repeat length of the tumor and normal sample. The proportion of unstable loci may then be fed into a logistic regression classifier.

In exemplary embodiments, the techniques for determining MSI include a sequencing data pre-processing process and an MSI status calling process. These processes may be applied to specific microsatellite regions, in particular a specific panel chromosomes with identified microsatellite regions.

In exemplary embodiments, an initial procedure includes sequencing data pre-processing. In particular, the methods and systems described herein may be used on information generated from next generation sequencing (NGS) techniques. Extracted DNA from tumor tissue is single or paired-end sequenced using a NGS platform, such as a platform offered by Illumina. Methods for sequencing using an NGS platform are described in further detail in, for instance, U.S. Patent Publication No. US20160085910A1, which is incorporated by reference in its entirety.

The results of sequencing (herein, the “raw sequencing data”) may be passed through a bioinformatics pipeline where the raw sequencing data is analyzed. After sequencing information is run through the bioinformatics pipeline, the sequencing information may be evaluated for quality control, e.g., through use of an automated quality control system. If the sample does not pass an initial quality control step, it may be manually reviewed. If the sample passes an automated quality control system or is manually passed, an alert may be published to a message bus that is configured to listen for messages from quality control systems. This message may contain sample identifiers, as well as the location of BAM files, i.e. a binary format for storing sequence data. When a message notifying that the topic is received, an MSI micro-service may be triggered. In one embodiment, the MSI micro-service launches a Jenkins job, which deploys an EC2 instance with an MSI Algorithm Docker image that may be stored in in an elastic container repository, such as the web server AWS ECR.

In exemplary embodiments, the techniques for determining MSI further include a process of MSI calling. In an example, a plurality of microsatellites is analyzed to determine the frequency of DNA slippage events. A “DNA slippage event” is a change in the length of repetitive regions in the genome, like microsatellites, due to local mismatches between DNA strands during replication. When the mismatch repair machinery is defective, these slippage events accumulate throughout the genome, particularly in microsatellite regions. For MSI testing, microsatellites may be selected on the basis of their instability in tumors with mismatch repair deficiencies, where microsatellites with greater instability are better candidates for selection.

In an example, the frequency of microsatellite instability is measured by obtaining the lengths of the microsatellite repeats for all reads that map to each locus and comparing that distribution of repeat lengths to the distribution of repeat lengths obtained from a matched normal sample at each locus using a statistical method, such as Kolmogorov-Smirnov test. A threshold of significance number is set, such as a false discovery rate of <=0.05, for the locus to determine whether it is considered unstable versus stable.

In an example, some or all of the 43 microsatellites listed in Table 1 may be used to determine the frequency of DNA slippage events. The information detected is provided to an MSI classification algorithm, described hereinbelow, which then classifies tumors into three categories: microsatellite instability high (MSI-H), microsatellite stable (MSS), or microsatellite equivocal (MSE). Table 1 illustrates the chromosome number, start and end position of the microsatellite, and the nucleotide or nucleotides repeated in that region of DNA (repeat unit).

Table 1 lists chromosomes with identified microsatellite regions. The first column lists the chromosome name. The second column lists the start position (genomic coordinates) of the microsatellite region (locus) within the chromosome. The third column lists the end position (genomic coordinates) of the microsatellite region (locus) within the chromosome. The fourth column lists the unit(s) that repeat throughout the microsatellite region.

TABLE 1
xT Microsatellite Regions

| Chromosome | MS start | MS end | Repeat unit |
|---|---|---|---|
| 18 | 649879 | 649893 | T |
| 14 | 73959703 | 73959718 | T |
| 8 | 23712066 | 23712077 | T |
| 3 | 71008341 | 71008353 | T |
| 4 | 39501722 | 39501739 | A |
| 3 | 113377481 | 113377491 | T |
| 14 | 53513439 | 53513450 | A |
| 19 | 14104688 | 14104701 | T |
| 11 | 63149670 | 63149680 | A |
| 6 | 11714639 | 11714652 | A |
| 1 | 47325299 | 47325311 | A |
| 3 | 130733046 | 130733056 | T |
| 3 | 30691871 | 30691880 | A |
| 13 | 58299434 | 58299444 | A |
| 11 | 115047032 | 115047045 | T |
| 1 | 149900985 | 149901000 | A |
| X | 101409254 | 101409269 | T |
| 8 | 79629738 | 79629751 | A |
| 11 | 125763610 | 125763622 | T |
| 12 | 85285920 | 85285936 | A |
| 5 | 67584512 | 67584523 | T |
| 3 | 164905648 | 164905658 | A |
| 21 | 33974094 | 33974107 | T |
| 5 | 58270457 | 58270468 | A |
| 2 | 211179765 | 211179775 | T |
| 1 | 237060945 | 237060959 | T |
| 6 | 71571694 | 71571705 | A |
| 3 | 123332875 | 123332890 | T |
| 18 | 319944 | 319954 | T |
| 2 | 64069277 | 64069291 | A |
| 12 | 13364426 | 13364435 | A |
| 5 | 88018388 | 88018400 | A |
| 21 | 30339205 | 30339215 | T |
| 17 | 1326754 | 1326767 | A |
| 5 | 161494990 | 161495001 | A |
| 15 | 37391752 | 37391763 | T |
| 6 | 43021976 | 43021987 | G |
| 7 | 94989255 | 94989271 | T |
| 2 | 99614595 | 99614607 | A |
| 2 | 152236045 | 152236058 | A |
| 11 | 65268479 | 65268490 | T |
| 2 | 118854094 | 118854106 | T |
| 12 | 31435608 | 31435618 | T |
| 1 | 89449508 | 89449518 | T |
| 2 | 148683685 | 148683692 | A |
| 1 | 33402334 | 33402350 | A |
| 4 | 83785564 | 83785572 | T |
| 11 | 77784146 | 77784154 | A |
| 7 | 30673512 | 30673526 | A |
| 4 | 158142151 | 158142159 | A |
| 18 | 9954233 | 9954245 | T |
| 12 | 130883554 | 130883566 | T |
| 19 | 9361740 | 9361750 | T |
| 8 | 33356825 | 33356837 | T |
| 1 | 221875661 | 221875671 | A |
| 17 | 55016354 | 55016367 | A |
| 12 | 54405179 | 54405189 | C |
| 1 | 6257784 | 6257791 | T |
| 2 | 197531518 | 197531528 | A |
| 19 | 12574740 | 12574760 | A |
| 9 | 110093970 | 110093981 | A |
| X | 18183097 | 18183111 | A |
| 1 | 159032486 | 159032495 | T |
| 18 | 30913142 | 30913151 | T |
| 20 | 58497481 | 58497495 | A |
| 22 | 39079920 | 39079934 | T |
| 17 | 56435160 | 56435166 | C |
| 7 | 54819993 | 54820003 | A |
| 2 | 88056970 | 88056980 | A |
| X | 101138438 | 101138448 | T |
| 5 | 79970914 | 79970921 | A |
| 20 | 52491773 | 52491783 | A |
| 9 | 86584222 | 86584232 | A |
| 12 | 118588761 | 118588778 | T |
| 19 | 44662274 | 44662286 | A |
| 6 | 167453519 | 167453530 | T |
| 1 | 116655183 | 116655195 | A |
| 20 | 58587783 | 58587792 | A |
| 6 | 49459994 | 49460003 | T |
| 6 | 55739711 | 55739724 | A |
| 11 | 126137086 | 126137093 | T |
| 2 | 8998775 | 8998786 | A |
| 6 | 88853530 | 88853540 | A |
| 5 | 158526534 | 158526548 | A |
| 10 | 74653468 | 74653477 | A |
| 5 | 82937251 | 82937260 | A |
| 13 | 47345469 | 47345482 | T |
| 18 | 57013193 | 57013201 | T |
| 3 | 170715683 | 170715692 | T |
| 11 | 104879686 | 104879695 | T |
| 1 | 28785729 | 28785738 | A |
| 17 | 4442639 | 4442656 | A |
| 8 | 38961268 | 38961278 | T |
| 11 | 104878040 | 104878049 | T |
| 2 | 114500276 | 114500285 | A |
| 3 | 124951171 | 124951185 | T |
| 11 | 5687320 | 5687336 | A |
| 1 | 161091814 | 161091830 | A |
| 8 | 33356191 | 33356206 | T |
| 17 | 33288433 | 33288443 | A |
| 7 | 151874147 | 151874155 | T |
| 14 | 45716018 | 45716028 | T |
| 3 | 111831723 | 111831735 | A |
| 20 | 44470648 | 44470660 | T |
| 6 | 71508369 | 71508378 | A |
| 5 | 16474778 | 16474793 | T |
| 3 | 12571420 | 12571432 | T |
| 5 | 145647319 | 145647327 | A |
| 19 | 52249071 | 52249084 | T |
| 2 | 179330468 | 179330478 | A |
| 10 | 78646910 | 78646918 | A |
| 1 | 160589600 | 160589608 | T |
| 10 | 27037487 | 27037497 | A |
| 10 | 121335156 | 121335164 | T |
| 2 | 55863360 | 55863370 | A |
| 1 | 153516162 | 153516171 | A |
| 1 | 23636874 | 23636884 | T |
| 15 | 25319287 | 25319302 | T |
| 4 | 177100754 | 177100764 | T |
| 3 | 98518160 | 98518169 | A |
| 11 | 18231788 | 18231797 | T |
| 14 | 88934423 | 88934431 | T |
| 3 | 44373517 | 44373531 | T |
| 9 | 27062802 | 27062814 | A |
| 11 | 26581382 | 26581421 | AC |
| 14 | 69520518 | 69520530 | T |
| 5 | 121362852 | 121362862 | A |
| 15 | 83659295 | 83659308 | T |
| 12 | 42835328 | 42835342 | A |
| 20 | 47858503 | 47858510 | A |
| 7 | 27868483 | 27868499 | A |
| 1 | 152195728 | 152195738 | T |
| 11 | 55065031 | 55065039 | T |
| X | 132351002 | 132351016 | A |
| 1 | 236714292 | 236714309 | A |
| 14 | 95999676 | 95999687 | A |
| 19 | 11944947 | 11944954 | T |
| 19 | 49850472 | 49850479 | G |
| 3 | 112719791 | 112719806 | A |
| 4 | 70160632 | 70160642 | A |
| 3 | 136573485 | 136573493 | A |
| 3 | 160395092 | 160395100 | A |
| 2 | 138721942 | 138721958 | T |
| 15 | 45848230 | 45848245 | T |
| 16 | 83828683 | 83828693 | A |
| 14 | 58471378 | 58471388 | T |
| 5 | 134086670 | 134086682 | A |
| X | 15364158 | 15364167 | T |
| 11 | 112832276 | 112832285 | G |
| 21 | 35475614 | 35475629 | A |
| 9 | 100700505 | 100700515 | A |
| X | 153906377 | 153906392 | A |
| 1 | 14108748 | 14108756 | A |
| 18 | 32917176 | 32917187 | T |
| 5 | 122359467 | 122359478 | A |
| 2 | 112252632 | 112252643 | A |
| 14 | 64990700 | 64990712 | T |
| 11 | 5799651 | 5799660 | A |
| 15 | 56736722 | 56736731 | T |
| 6 | 54130829 | 54130842 | T |
| 11 | 134188851 | 134188865 | T |
| 1 | 78414310 | 78414327 | A |
| 8 | 103289348 | 103289355 | T |
| 12 | 55038408 | 55038430 | T |
| 8 | 18393159 | 18393169 | T |
| 11 | 63886066 | 63886077 | T |
| 17 | 1248583 | 1248600 | A |
| 3 | 194409257 | 194409271 | A |
| X | 135961587 | 135961599 | T |
| 2 | 173368989 | 173368999 | A |
| 9 | 114694383 | 114694392 | A |
| 6 | 108214754 | 108214763 | T |
| 15 | 83677270 | 83677283 | A |
| 4 | 9785354 | 9785364 | A |
| 9 | 20346377 | 20346390 | A |
| 10 | 90034667 | 90034677 | A |
| 9 | 20346393 | 20346404 | A |
| 10 | 75135833 | 75135845 | A |
| X | 151302890 | 151302908 | T |
| 5 | 162945387 | 162945398 | T |
| 11 | 36601096 | 36601107 | T |
| 5 | 134907432 | 134907474 | T |
| 11 | 93170909 | 93170917 | C |
| 4 | 56336953 | 56336961 | A |
| 1 | 118502087 | 118502097 | T |
| 15 | 48634319 | 48634333 | A |
| 9 | 129596297 | 129596307 | A |
| 14 | 70793015 | 70793028 | A |
| 7 | 77423459 | 77423467 | T |
| 1 | 110906247 | 110906265 | A |
| 4 | 57220268 | 57220277 | A |
| 19 | 21561408 | 21561426 | T |
| 8 | 38845644 | 38845656 | A |
| 4 | 93225752 | 93225763 | A |
| 12 | 64812754 | 64812762 | T |
| 13 | 115057210 | 115057218 | A |
| 19 | 38161161 | 38161171 | T |
| X | 37312610 | 37312617 | C |
| 1 | 33146113 | 33146123 | T |
| 11 | 43515493 | 43515501 | A |
| 10 | 124924484 | 124924494 | T |
| 5 | 122359502 | 122359531 | AC |

In exemplary embodiments, a MSI classification algorithm is applied to the sequencing data that has passed quality control. The algorithm may be performed in paired mode, where the algorithm has access to matched tumor-normal sequencing data. The algorithm may also be performed in unpaired mode, if the algorithm does not have access to paired normal sequencing data.

In an example of a MSI classification (i.e., detection) performed in a paired mode, initially MSI loci read filtering and sampling quality control is performed. For example, to be an MSI locus mapping read, the read must be mapped to the MSI locus during alignment with a bioinformatics pipeline, such as the Tempus xT bioinformatics pipeline. In an example, the mapping read must also contain at least 3-6 mapping base pairs in both the front and rear flank of the microsatellite, with any number of the expected repeating units in between.

In an example, at least 30-40 mapping reads in the tumor sample and 30-40 mapping reads in the normal sample must be identified for a microsatellite to be included in the analysis. This defines an example coverage minimum. Further, at least 20-30 of the 43 microsatellites on the panel must reach the coverage minimum described above for the assay to be run. If this coverage threshold is not met for the normal sample, MSI detection and calling will switch to running in unpaired mode, discussed further below.

With the coverage threshold met, MSI classification is performed. In an exemplary embodiment of MSI classification in the paired mode, each microsatellite is tested for instability. For example, each microsatellite locus may be tested for instability by measuring changes in the distribution of the number of repeat units in the tumor reads compared to the distribution of the number of repeat units in the normal reads. An example method of measurement for use is the statistical Kolmogorov-Smirnov test. If p<=0.05, the locus may be considered unstable. That is, a statistical analysis is performed that analyzes the distribution of reads mapping to a locus of a tumor sample with the distribution of reads mapping to a locus of a normal sample.

The proportion of unstable microsatellites per sample across all loci may then be provided to a univariate logistic regression classifier. In an example, the classifier already has been trained on data from cancer samples. For instance, the classifier may have been trained on data from colorectal and endometrial cohorts that have clinically determined MSI statuses from MSI PCR testing, such as cohorts from The Cancer Genome Atlas (“TCGA”, available from the U.S. National Institutes of Health, Bethesda, Md.). In an example training process, the same microsatellites used with present MSI test were assessed for instability in TCGA samples (e.g., 245 TCGA samples although training may be performed on fewer or larger numbers). The TCGA MSI PCR statuses were converted to a binary dependent variable: e.g., whether the sample was MSI-H or not. A logistic regression classifier was then trained to predict the binary MSI-H status using the proportion of unstable microsatellites. The output of the trained logistic function can then be interpreted as the probability of the dependent variable being categorized as MSI-H or not. To address different numbers of MSS and MSI-H samples in training set, the class weights were set to be inversely proportional to class frequencies (number of MSS and MSI-H samples) in the input data during training.

The classifier groups the samples into three categories: MSI-H, MSE, and MSS. If there is a greater than 70% probability of MSI-H status, the sample is classified as MSI-H. If there is between 50-70% probability of MSI-H status, the test results are too ambiguous to interpret. Those samples should make up a relatively small proportion of samples and are classified as MSE. If there is less than 50% probability of MSI-H status, the sample is considered MSS.

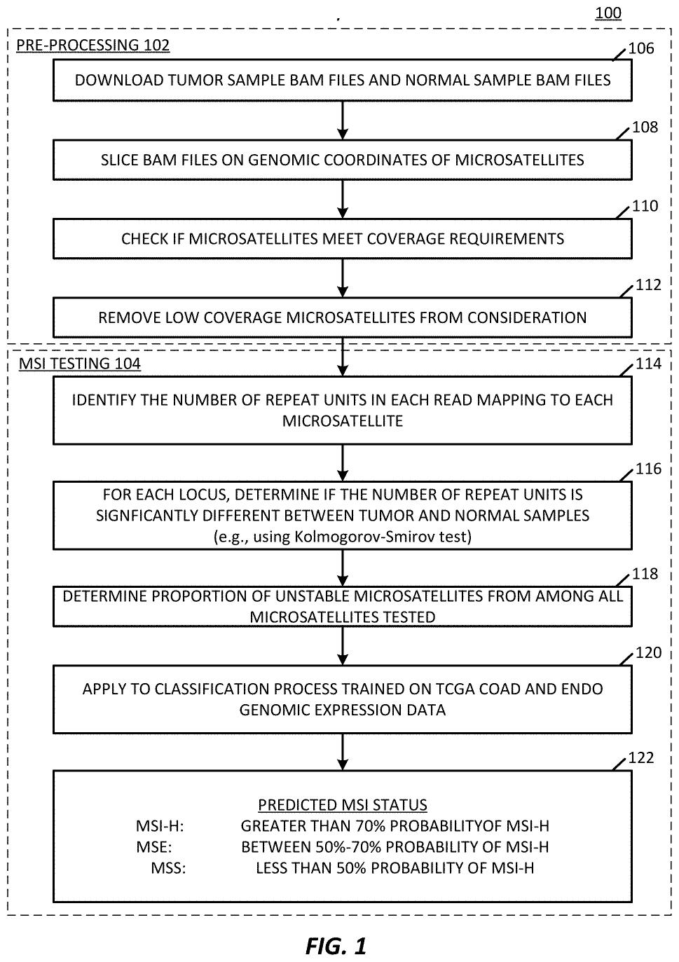

FIG. 1 illustrates an example of the MSI Detection and classification process in paired mode using tumor and normal matched samples, in accordance with an example. A process 100 includes a pre-processing procedure 102 and an MSI testing procedure 104. During the pre-processing procedure 102, at process 106, a MSI determination processing system electronically receives BAM files from a resource, such as a next generation sequencer, stored databased on gene expression data, or other resource coupled to the MSI determination processing system through a network or other interface. The processing system slices the BAM files on genomic coordinates of microsatellites, at process 108. To determine if suitable microsatellite data is available, the processing system, at a process 110, determines if the microsatellite data meets sufficient coverage requirements, such as covering a sufficient number of generic sequencing reads. In some examples, the process 110 may determine if the microsatellite data covers reads such as those corresponding to all or desired portion of Table 1 are covered. For any low coverage microsatellites, the processing system removes those low coverage microsatellites from consideration, at a process 112.

In the MSI testing procedure 104, at process 114, the MSI determination processing system identifies the number of repeat units in each read mapping to each microsatellite identified by process 108 and meeting the coverage requirements of process 110. For each locus, the processing system determines in the number of repeat units is significantly different between gene expression data from tumor samples and gene expression data from normal (non-tumor) samples, at process 116. In an example, the process 116 performs a statistical analysis, such as Kolmogorov-Smirov test, to determine if there is significant difference in gene expression data. For example, the process 116 may compare a mapping of a first set of genomic sequencing reads (such as reads onto a tumor sample) to a mapping of a second set of genomic sequencing reads (such as reads on a normal sample) using a Kolmogorov-Smirov test. The proportion of unstable microsatellites from among all the microsatellites tested at process 114 is determined at process 118, for example applying instability determination techniques described herein, such as those based on the Kolmogorov-Smirov test.

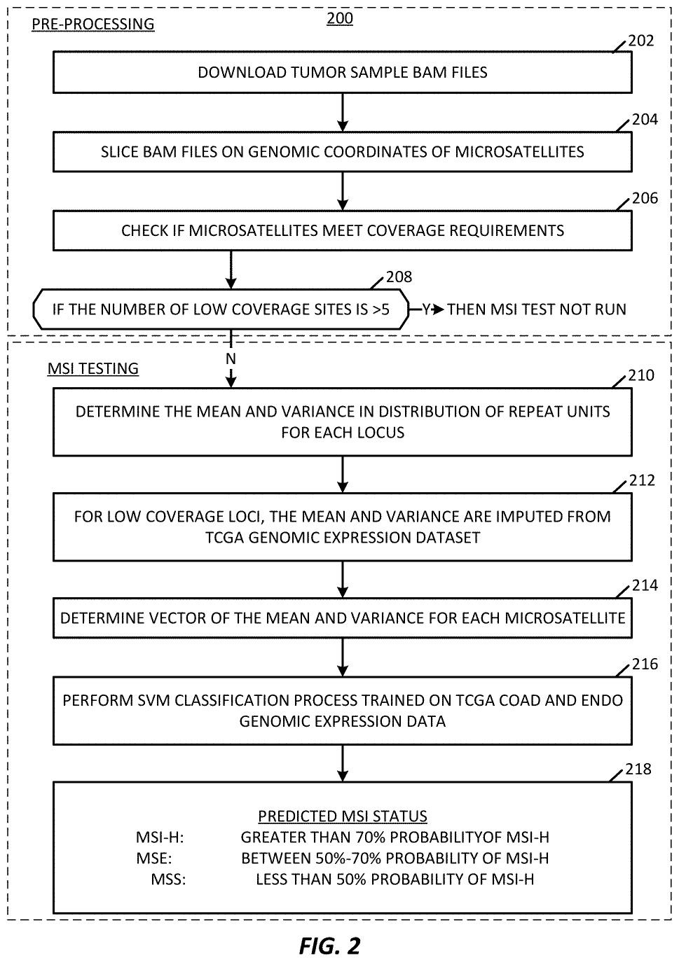

At a process 120, which may also be performed by the MSI determination processing system, the repeat units and comparison data from the process 118 is provided to a trained MSI classifier which determines a predicted MSI status generates a predicted MSI status report at process 122. As discussed herein, in this paired mode, the MSI classification at process 120 may be performed each microsatellite, testing each microsatellite for instability. The trained classifier, in the illustrated example, is trained on genomic expression data from the TCGA dataset, and in particular genomic expression data on colon adenocarcinoma (COAD) tumor samples and endometrial (ENDO) cohorts samples, that are used for determine MSI status of suitable tissue samples. In various embodiments, the training data for the classifier includes DNA sequencing data for the microsatellite regions used in the MSI assay paired with the MSI status of the tumor. In another exemplary embodiment, a MSI classification algorithm is applied to the sequencing data an in unpaired mode using tumor-only samples, as shown in FIG. 2 and as may be implemented on an MSI determination processing system. MSI detection and calling process 200, which is configured as an unpaired mode, is used for tumor-only samples, i.e., where there is no matched tumor-normal sequencing data at process 202, or if the coverage threshold discussed above is not met for the normal sample in paired mode. The received tumor sample BAM files are sliced on genomic coordinates of microsatellites at process 204, similar to process 108 of FIG. 1. Similarly, the processing system performs a check to see if microsatellite slicing meets coverage requirements, at a process 206.

As in the paired mode, initially MSI loci read filtering and sampling quality control is performed. To be a MSI loci mapping read, the read must be mapped to the MSI locus during the alignment process of a bioinformatics pipeline. In an example, a process 208 determines if sufficient microsatellite coverage data exists to perform MSI testing. In an example, at the process 208 determines if there is sufficient microsatellite coverage by looking at the front and rear flank of the microsatellite and determining if a threshold number of base pairs appear at both the front and rear flank. For example, the process 208 may be configured such that the mapping read is to contain the 5 base pairs in both the front and rear flank of the microsatellite, with any number of expected repeating unit in between. In this example, if 5 or more microsatellites have less than 30× coverage, the assay cannot be run.

In an exemplary embodiment of MSI classification in the unpaired mode, the MSI testing process receives the microsatellite coverage data, and at a process 210 determines the mean and variance of the distribution of the number of repeat units, which is calculated for each microsatellite locus in a sample. If there are no reads mapping to a particular locus, the mean and variance of the number of repeat units is imputed for that locus based on the average values from the tumors in a training set, such as the TCGA training data, at a process 212. In an example, if at least 20-30 microsatellites do not meet the coverage minimum when mapping the second plurality of genomic sequencing reads, then the process 212 may replace the mapping of the second plurality of genomic sequencing reads with mean and variance data from trained sequencing data before performing the classification.

In the illustrated example, a vector containing the mean and variance data for each microsatellite locus (provided at process 214) is put into a support vector machine (SVM) classification algorithm (process 216), with a linear kernel trained on samples from the TCGA colorectal and endometrial cohorts that have clinically determined MSI statuses. In an example, the mean and variance of the repeat length for each microsatellite was determined for all the TCGA training samples and the corresponding MSI PCR statuses were converted to a binary dependent variable representing whether the sample was MSI-H or not. A SVM was then trained to predict the binary MSI-H status using the mean and variance data. When running patient samples, Platt scaling is used to transform the outputs of the SVM classifier into a probability distribution over classes, returning the probability of the patient being MSI-H.

The trained MSI SVM classification algorithm groups samples into three categories: MSI-H, MSE, and MSS, and generates a report at process 218. If there is a greater than 70% probability of MSI-H status, the sample is classified as MSI-H. If there is between 50-70% probability of MSI-H status, the test results is too ambiguous to interpret. Those samples should make up a relatively small proportion of samples and are classified as MSE. If there is less than 50% probability of MSI-H status, the sample is considered MSS. These thresholds were generated after evaluation of samples that received both the MSI detection and calling, as well as an orthogonal clinically validated MSI test.

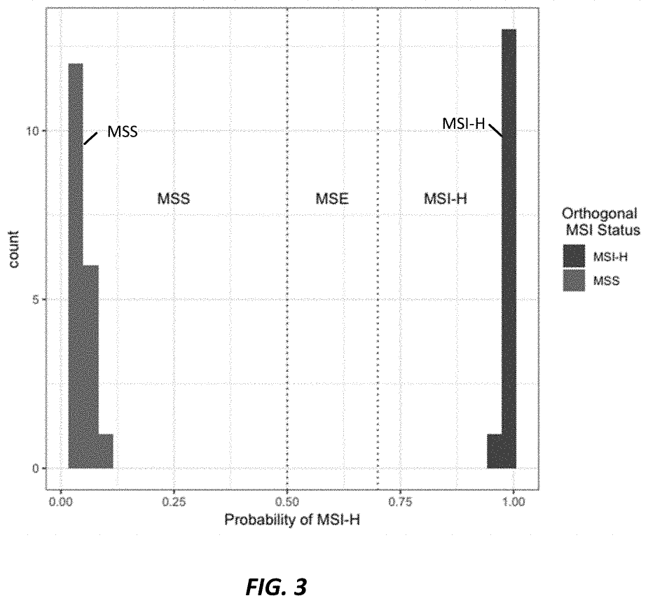

FIG. 3 displays a graph of validation results for microsatellite status classification from a genomic sequencing assay on a set of tumor samples. The graph displays the count of samples (y-axis) and exemplary thresholds of MSI-H, MSE, and MSS (x-axis). If there is a greater than 70% probability of MSI-H status, the sample is classified as MSI-H. If there is between 50-70% probability of MSI-H status, the test results is too ambiguous to interpret and is classified as MSE. If there is less than 50% probability of MSI-H status, the sample is considered MSS.

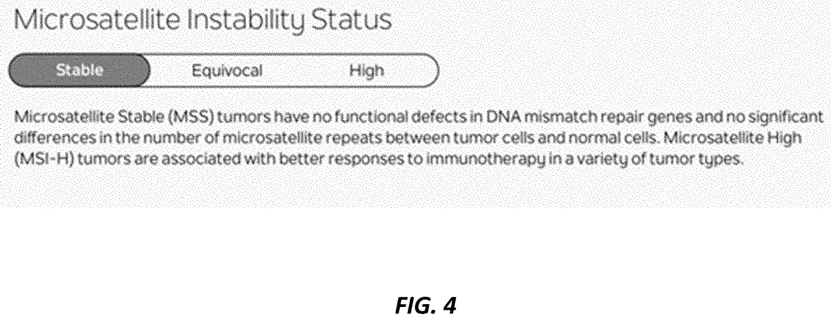

After MSI detection and calling is performed, for example in a cloud based server on the EC2 instance, the results may be written and saved to a network-connected production database, a network-connected immunotherapy research database, and the logs may be stored in S3. Results may be sent to physician in a printable report, digital online portal, and other media forms, such as a digital PDF or mobile application. FIG. 4 illustrates an example clinical digital report displaying MSI status to physicians. In this example, the patient was MSI “Stable” (i.e., MSS) and had less than 50% probability of MSI-H status as illustrated in FIG. 3. If the MSI status was MSE, then the “Equivocal” indication would be highlighted in the displayed report; and corresponding if the MSI status was MSI-H, then the “High” indication would be highlighted.

With the MSI classification, in some examples, the techniques herein further include therapy matching based on the MSI classification. That is, the outcome of the techniques described herein is useful, for example, for determining appropriate treatment regimens for cancer patients. For instance, immune checkpoint inhibitors are suitable for treating cancers with microsatellite instability (MSI). Pembrolizumab (KEYTRUDA, Merck & Co.), for example, can be administered to adult and pediatric patients with unresectable or metastatic, microsatellite instability-high (MSI-H) or mismatch repair deficient (dMMR) solid tumors, including in those patients that have progressed following prior treatment and who have no satisfactory alternative treatment options. Pembrolizumab also may be administered to patients with MSI-H or dMMR colorectal cancer that has progressed following treatment with a fluoropyrimidine, oxaliplatin, and irinotecan. Ipilimumab (YERVOY, Bristol-Myers Squibb Company Inc.) and nivolumab (OPDIVO, Bristol-Myers Squibb Company) can be administered, for example, in MSI-H or dMMR metastatic colorectal cancer (mCRC) patients, including patients that have progressed following treatment with a fluoropyrimidine, oxaliplatin, and irinotecan. An example of an anti-PD-L1 antibody is durvalumab. An example of an anti-CTLA antibody is tremelimumab. In various aspects, the disclosure contemplates a method wherein a cancer therapy, such as a checkpoint inhibitor (e.g., PD-1 inhibitor, PD-L1 inhibitor, PD-L2 inhibitor, CTLA-4 inhibitor, and the like), is administered to patient with MSI-H tumors as determined by the methods described herein.

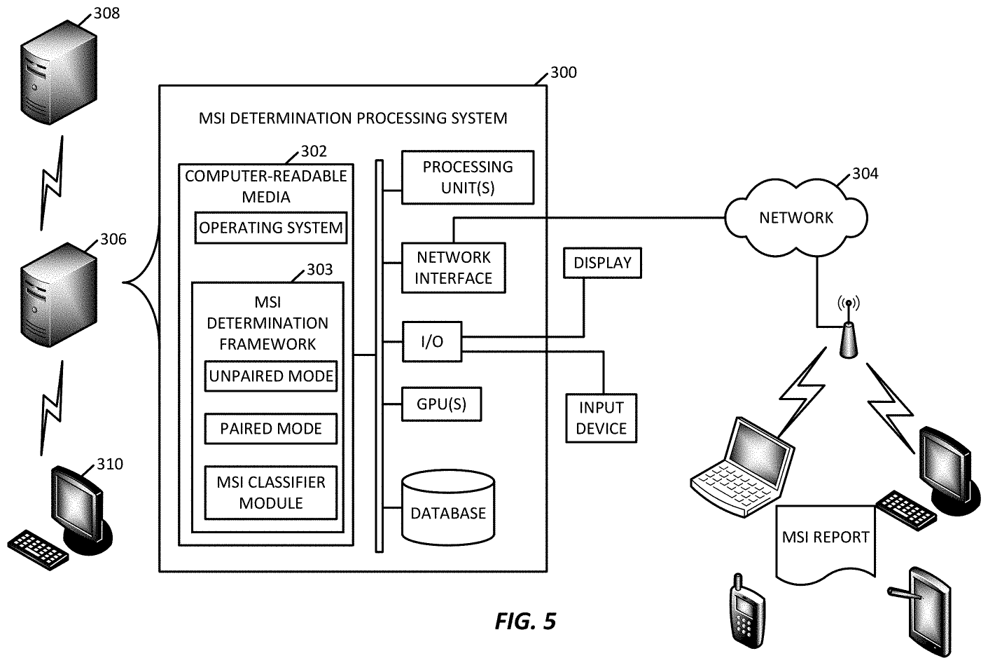

FIG. 5 illustrates an MSI determination processing system 300 that may be implemented on a computing device such as a computer, tablet or other mobile computing device, or server. The system 300 may include a number of processors, controllers or other electronic components for processing sequence data and performing the processes described herein. As illustrated, the system 300 may be implemented on a computing device and in particular on one or more processing units, which may represent Central Processing Units (CPUs), and/or on one or more or Graphical Processing Units (GPUs), including clusters of CPUs and/or GPUs. Features and functions described for the system 300 may be stored on and implemented from one or more non-transitory computer-readable media 302 of the computing device.

The computer-readable media 302 may include, for example, an operating system and an MSI determination framework 303 having elements configured to perform the processes described herein, including those of FIGS. 1 and 2. For example, the MSI determination framework 303 may include an unpaired mode process controller for executing the process of FIG. 2 and a paired mode process controller for executing the process of FIG. 1. Each of these controls may access an MSI classifier module that may include trained paired mode classifiers and trained unpaired mode classifiers. More generally, the computer-readable media 302 may store any number of trained classifiers, such as SVM models, executable code, etc. for implementing the techniques herein. The processing system 300 includes a network interface communicatively coupled to a network 304, for communicating to and/or from a portable personal computer, smart phone, electronic document, tablet, and/or desktop personal computer, or other computing devices. The processing system 300 further includes an I/O interface connected to devices, such as digital displays, user input devices, etc. In some examples, the processing system 300 generates MSI prediction status reports, like that of FIG. 4, that are displayed on the digital displays connected through an I/O interface or that are communicated to remote connected processing devices through the network 304 for display, as shown.

In some examples, the MSI determination processing system 300 is configured to additionally report a therapeutic option corresponding to the predicted MSI status determined by the techniques herein. For example, based on the MSI status, the processing system 300 may generate a list of matched possible therapies, from among a plurality of available therapies. Possible therapeutic options that may be reported include any one of fluoropyrimidine, oxaliplatin, irinotecan, Ipilimumab, nivolumab, Pembrolizumab, an anti-PD-L1 antibody (e.g., durvalumab), an anti-CTLA antibody (e.g., tremelimumab), and checkpoint inhibitor (e.g., PD-1 inhibitor, PD-L1 inhibitor, PD-L2 inhibitor, CTLA-4 inhibitor). For example, to treat a subject with nivolumab or Pembrolizumab an MSI-H status prediction may be required; therefore a determined MSI-H status may result in the processing system 300 identifying these possible therapies. If other therapies are possible based on the MSI status, then the processing system 300 may determine and generate a reporting of a more expansive list of possible therapies.

In the illustrated example, the processing system 300 is implemented on a single server 306. However, the functions of the processing system 300 may be implemented across distributed devices 306, 308, 310, etc. connected to one another through a communication link. In other examples, functionality of the processing system 300 may be distributed across any number of devices, including the portable personal computer, smart phone, electronic document, tablet, and desktop personal computer devices shown. The network 304 may be a public network such as the Internet, private network such as research institutions or corporations private network, or any combination thereof. Networks can include, local area network (LAN), wide area network (WAN), cellular, satellite, or other network infrastructure, whether wireless or wired. The network can utilize communications protocols, including packet-based and/or datagram-based protocols such as internet protocol (IP), transmission control protocol (TCP), user datagram protocol (UDP), or other types of protocols. Moreover, the network can include a number of devices that facilitate network communications and/or form a hardware basis for the networks, such as switches, routers, gateways, access points (such as a wireless access point as shown), firewalls, base stations, repeaters, backbone devices, etc.

The computer-readable media 302 may include executable computer-readable code stored thereon for programming a computer (e.g., comprising a processor(s) and GPU(s)) to the techniques herein. Examples of such computer-readable storage media include a hard disk, a CD-ROM, digital versatile disks (DVDs), an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. More generally, the processing units of the computing device 102 may represent a CPU-type processing unit, a GPU-type processing unit, a field-programmable gate array (FPGA), another class of digital signal processor (DSP), or other hardware logic components that can be driven by a CPU.

Throughout this specification, plural instances may implement components, operations, or structures described as a single instance. Although individual operations of one or more methods are illustrated and described as separate operations, one or more of the individual operations may be performed concurrently, and nothing requires that the operations be performed in the order illustrated. Structures and functionality presented as separate components in example configurations may be implemented as a combined structure or component. Similarly, structures and functionality presented as a single component may be implemented as separate components or multiple components. These and other variations, modifications, additions, and improvements fall within the scope of the subject matter herein.

Additionally, certain embodiments are described herein as including logic or a number of routines, subroutines, applications, or instructions. These may constitute either software (e.g., code embodied on a machine-readable medium or in a transmission signal) or hardware. In hardware, the routines, etc., are tangible units capable of performing certain operations and may be configured or arranged in a certain manner. In example embodiments, one or more computer systems (e.g., a standalone, client or server computer system) or one or more hardware modules of a computer system (e.g., a processor or a group of processors) may be configured by software (e.g., an application or application portion) as a hardware module that operates to perform certain operations as described herein.

In various embodiments, a hardware module may be implemented mechanically or electronically. For example, a hardware module may comprise dedicated circuitry or logic that is permanently configured (e.g., as a special-purpose processor, such as a microcontroller, field programmable gate array (FPGA) or an application-specific integrated circuit (ASIC)) to perform certain operations. A hardware module may also comprise programmable logic or circuitry (e.g., as encompassed within a general-purpose processor or other programmable processor) that is temporarily configured by software to perform certain operations. It will be appreciated that the decision to implement a hardware module mechanically, in dedicated and permanently configured circuitry, or in temporarily configured circuitry (e.g., configured by software) may be driven by cost and time considerations.

Accordingly, the term “hardware module” should be understood to encompass a tangible entity, be that an entity that is physically constructed, permanently configured (e.g., hardwired), or temporarily configured (e.g., programmed) to operate in a certain manner or to perform certain operations described herein. Considering embodiments in which hardware modules are temporarily configured (e.g., programmed), each of the hardware modules need not be configured or instantiated at any one instance in time. For example, where the hardware modules comprise a general-purpose processor configured using software, the general-purpose processor may be configured as respective different hardware modules at different times. Software may accordingly configure a processor, for example, to constitute a particular hardware module at one instance of time and to constitute a different hardware module at a different instance of time.

Hardware modules can provide information to, and receive information from, other hardware modules. Accordingly, the described hardware modules may be regarded as being communicatively coupled. Where multiple of such hardware modules exist contemporaneously, communications may be achieved through signal transmission (e.g., over appropriate circuits and buses) that connects the hardware modules. In embodiments in which multiple hardware modules are configured or instantiated at different times, communications between such hardware modules may be achieved, for example, through the storage and retrieval of information in memory structures to which the multiple hardware modules have access. For example, one hardware module may perform an operation and store the output of that operation in a memory device to which it is communicatively coupled. A further hardware module may then, at a later time, access the memory device to retrieve and process the stored output. Hardware modules may also initiate communications with input or output devices, and can operate on a resource (e.g., a collection of information).

The various operations of the example methods described herein can be performed, at least partially, by one or more processors that are temporarily configured (e.g., by software) or permanently configured to perform the relevant operations. Whether temporarily or permanently configured, such processors may constitute processor-implemented modules that operate to perform one or more operations or functions. The modules referred to herein may, in some example embodiments, comprise processor-implemented modules.

Similarly, the methods or routines described herein may be at least partially processor-implemented. For example, at least some of the operations of a method can be performed by one or more processors or processor-implemented hardware modules. The performance of certain of the operations may be distributed among the one or more processors, not only residing within a single machine, but also deployed across a number of machines. In some example embodiments, the processor or processors may be located in a single location (e.g., within a home environment, an office environment or as a server farm), while in other embodiments the processors may be distributed across a number of locations.

The performance of certain of the operations may be distributed among the one or more processors, not only residing within a single machine, but also deployed across a number of machines. In some example embodiments, the one or more processors or processor-implemented modules may be located in a single geographic location (e.g., within a home environment, an office environment, or a server farm). In other example embodiments, the one or more processors or processor-implemented modules may be distributed across a number of geographic locations.

Unless specifically stated otherwise, discussions herein using words such as “processing,” “computing,” “calculating,” “determining,” “presenting,” “displaying,” or the like may refer to actions or processes of a machine (e.g., a computer) that manipulates or transforms data represented as physical (e.g., electronic, magnetic, or optical) quantities within one or more memories (e.g., volatile memory, non-volatile memory, or a combination thereof), registers, or other machine components that receive, store, transmit, or display information.

As used herein any reference to “one embodiment” or “an embodiment” means that a particular element, feature, structure, or characteristic described in connection with the embodiment is included in at least one embodiment. The appearances of the phrase “in one embodiment” in various places in the specification are not necessarily all referring to the same embodiment.

Some embodiments may be described using the expression “coupled” and “connected” along with their derivatives. For example, some embodiments may be described using the term “coupled” to indicate that two or more elements are in direct physical or electrical contact. The term “coupled,” however, may also mean that two or more elements are not in direct contact with each other, but yet still co-operate or interact with each other. The embodiments are not limited in this context.

As used herein, the terms “comprises,” “comprising,” “includes,” “including,” “has,” “having” or any other variation thereof, are intended to cover a non-exclusive inclusion. For example, a process, method, article, or apparatus that comprises a list of elements is not necessarily limited to only those elements but may include other elements not expressly listed or inherent to such process, method, article, or apparatus. Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

In addition, use of the “a” or “an” are employed to describe elements and components of the embodiments herein. This is done merely for convenience and to give a general sense of the description. This description, and the claims that follow, should be read to include one or at least one and the singular also includes the plural unless it is obvious that it is meant otherwise.

This detailed description is to be construed as an example only and does not describe every possible embodiment, as describing every possible embodiment would be impractical, if not impossible. One could implement numerous alternate embodiments, using either current technology or technology developed after the filing date of this application.

What is claimed:

1. A computer-implemented method of indicating a likelihood of microsatellite instability, the method comprising:
for each locus in a plurality of microsatellite instability (MSI) loci:
mapping a first plurality of genomic sequencing reads from a tumor specimen to the locus;
mapping a second plurality of genomic sequencing reads from a matched-normal specimen to the locus;
comparing the mapping of the first plurality to the mapping of the second plurality and determining the likelihood of microsatellite instability based on the comparison; and
generating a report indicating the determined likelihood of microsatellite instability.

2. The computer-implemented method of claim 1, wherein the plurality of MSI loci includes at least one locus listed in Table 1.

3. The computer-implemented method of claim 1, wherein the plurality of MSI loci includes all of the loci listed in Table 1.

4. The computer-implemented method of claim 1, wherein the plurality of MSI loci includes at least one locus on a chromosome listed in Table 1.

5. The computer-implemented method of claim 1, wherein each locus in the plurality of MSI loci is positioned on a chromosome listed in Table 1.

6. The computer-implemented method of claim 1, wherein the mapping the first plurality comprises mapping reads containing 3-6 base pairs, and wherein the mapping the second plurality comprises mapping reads containing 3-6 base pairs

7. The computer-implemented method of claim 1, wherein mapping the first plurality of genomic sequencing reads comprises mapping at least 30-40 genomic sequencing reads from the tumor sample; and wherein mapping the second plurality of genomic sequencing reads comprises mapping at least 30-40 genomic sequencing reads from the normal sample.

8. The computer-implemented method of claim 7, further comprising:
when mapping the first plurality of genomic sequencing reads, determining if at least 20-30 microsatellites meet a coverage minimum; and
when mapping the second plurality of genomic sequencing reads, determining if at least 20-30 microsatellites meet a coverage minimum.

9. The computer-implemented method of claim 8, further comprising: if at least 20-30 microsatellites do not meet the coverage minimum when mapping the second plurality of genomic sequencing reads, then replacing the mapping of the second plurality of genomic sequencing reads with mean and variance data from a trained sequencing data before performing the comparison.

10. The computer-implemented method of claim 1, further comprising comparing the mapping of the first plurality to the mapping of the second plurality and determining the likelihood of microsatellite instability based on the comparison by measuring changes in the number of repeat units in the first plurality of genomic sequencing reads from the tumor specimen to the number of repeat units in the second plurality of genomic sequencing reads from the matched-normal specimen

11. The computer-implemented method of claim 1, further comprising comparing the mapping of the first plurality to the mapping of the second plurality and determining the likelihood of microsatellite instability based on the comparison using a Kolmogorov-Smirov test.

12. The computer-implemented method of claim 11, further comprising determining the likelihood of microsatellite instability based on a p value.

13. The computer-implemented method of claim 1, further comprising: determining the likelihood of microsatellite instability as microsatellite instability high (MSI-H), microsatellite stable (MSI-S), or microsatellite equivocal (MSI-E).

14. The computer-implemented method of claim 13, wherein MSI-H is > about 70% probability, MSI-E is between about 50% and about 70% probability, and MSI-S is < about 50%, where “about” is defined as between 0% to 10% +/− difference.

15. The computer-implemented method of claim 1, further comprising determining a therapeutic for a subject based on the determined likelihood of microsatellite instability.

16. The computer-implemented method of claim 15, wherein the therapeutic is selected from the group consisting of fluoropyrimidine, oxaliplatin, irinotecan, Ipilimumab, nivolumab, Pembrolizumab, an anti-PD-L1 antibody (e.g., durvalumab), an anti-CTLA antibody (e.g., tremelimumab), and checkpoint inhibitor (e.g., PD-1 inhibitor, PD-L1 inhibitor, PD-L2 inhibitor, CTLA-4 inhibitor).

17. A computing device configured to indicate a likelihood of microsatellite instability, the computing device comprising one or more processors configured to:
for each locus in a plurality of microsatellite instability (MSI) loci:
map a first plurality of genomic sequencing reads from a tumor specimen to the locus;
map a second plurality of genomic sequencing reads from a matched-normal specimen to the locus;
compare the mapping of the first plurality to the mapping of the second plurality and determine the likelihood of microsatellite instability based on the comparison; and
generate a report indicating the determined likelihood of microsatellite instability.

18. The computing device of claim 17, wherein the plurality of MSI loci includes at least one locus listed in Table 1.

19. The computing device of claim 17, wherein the plurality of MSI loci includes all of the loci listed in Table 1.

20. The computing device of claim 17, wherein the plurality of MSI loci includes at least one locus on a chromosome listed in Table 1.

21. The computing device of claim 17, wherein each locus in the plurality of MSI loci is positioned on a chromosome listed in Table 1.

22. The computing device of claim 17, wherein the one or more processors are configured to map of the first plurality by mapping reads containing 3-6 base pairs, and wherein the one or more processors are configured to map the second plurality by mapping reads containing 3-6 base pairs

23. The computing device of claim 17, wherein the one or more processors are configured to map the first plurality of genomic sequencing reads by mapping at least 30-40 genomic sequencing reads from the tumor sample; and wherein the one or more processors are configured to map the second plurality of genomic sequencing reads by mapping at least 30-40 genomic sequencing reads from the normal sample.

24. The computing device of claim 23, wherein the one or more processors are further configured to:
when mapping the first plurality of genomic sequencing reads, determine if at least 20-30 microsatellites meet a coverage minimum; and
when mapping the second plurality of genomic sequencing reads, determine if at least 20-30 microsatellites meet a coverage minimum.

25. The computing device of claim 24, wherein the one or more processors are further configured to: if at least 20-30 microsatellites do not meet the coverage minimum when mapping the second plurality of genomic sequencing reads, then replace the mapping of the second plurality of genomic sequencing reads with mean and variance data from a trained sequencing data before performing the comparison.

26. The computing device of claim 17, wherein the one or more processors are further configured to: compare the mapping of the first plurality to the mapping of the second plurality and determine the likelihood of microsatellite instability based on the comparison by measuring changes in the number of repeat units in the first plurality of genomic sequencing reads from the tumor specimen to the number of repeat units in the second plurality of genomic sequencing reads from the matched-normal specimen

27. The computing device of claim 17, wherein the one or more processors are further configured to: compare the mapping of the first plurality to the mapping of the second plurality and determine the likelihood of microsatellite instability based on the comparison using a Kolmogorov-Smirov test.

28. The computing device of claim 27, wherein the one or more processors are further configured to: determine the likelihood of microsatellite instability based on a p value.

29. The computing device of claim 17, wherein the one or more processors are further configured to: determine the likelihood of microsatellite instability as microsatellite instability high (MSI-H), microsatellite stable (MSI-S), or microsatellite equivocal (MSI-E).

30. The computing device of claim 29, wherein MSI-H is > about 70% probability, MSI-E is between about 50% and about 70% probability, and MSI-S is < about 50%, where “about” is defined as between 0% to 10% +/− difference.

31. The computing device of claim 17, wherein the one or more processors are further configured to: determine a therapeutic for a subject based on the determined likelihood of microsatellite instability.

32. The computing device of claim 31, wherein the therapeutic is selected from the group consisting of fluoropyrimidine, oxaliplatin, irinotecan, Ipilimumab, nivolumab, Pembrolizumab, an anti-PD-L1 antibody (e.g., durvalumab), an anti-CTLA antibody (e.g., tremelimumab), and checkpoint inhibitor (e.g., PD-1 inhibitor, PD-L1 inhibitor, PD-L2 inhibitor, CTLA-4 inhibitor).
